(19) United States
(12) Patent Application Publication
(10) Pub. No.: US 2009/0004277 A1
(43) Pub. Date: Jan. 1, 2009

(54) Nanoparticle dispersion containing lactam compound

(76) Inventors: Miriam K. Franchini, Allentown, NJ (US); Thomas A. Haby, Hillsborough, NJ (US); Elaine Liversidge, West Chester, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/129,807

(22) Filed: May 16, 2005

Related U.S. Application Data
(60) Provisional application No. 60/572,279, filed on May 18, 2004.

(51) Int. Cl. A61K 31/427; A61K 9/14

(52) U.S. Cl. 424/489; 514/365

(57) ABSTRACT

Disclosed is a nanoparticle dispersion comprising nanoparticles dispersed in an aqueous medium in the presence of at least one stabilizer. The nanoparticles comprise at least lactam compound of formula I:

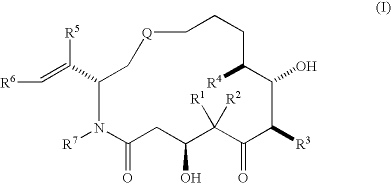

wherein R¹, R², R³, R⁴, R⁵, R⁶, R⁷, and Q are defined herein. A method is provided of the parenteral administration of the nanoparticle dispersion as a treatment for cancer or another proliferative disease. Also, a solid nanoparticulate composition comprising the nanoparticles of the lactam compound, and a method of administration are disclosed.

### CROSS-REFERENCE TO RELATED APPLICATIONS

The present application claims the priority benefit of U.S. Provisional Application No. 60/572,279, filed May 18, 2004, which is incorporated herein by reference in its entirety.

### FIELD OF THE INVENTION

The present invention generally relates to compositions comprising nanoparticles of one or more lactam compounds. The compositions are useful for the treatment of tumors. Methods of using the compositions to treat cancer or other proliferative diseases are also provided.

### BACKGROUND OF THE INVENTION

Epothilones are macrolide compounds having utility in the pharmaceutical field. For example, Epothilones A and B are naturally-occurring compounds that can be isolated from certain microorganisms, having the structures:

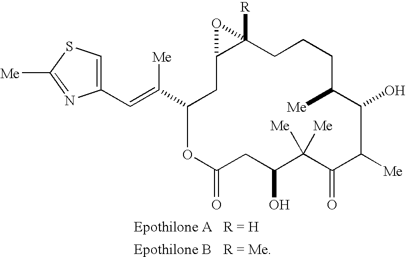

Known epothilones exert microtubule-stabilizing effects similar to TAXOL® and therefore exhibit cytotoxic activity against rapidly proliferating cells, such as occur in cancer and other hyperproliferative cellular diseases (See Angew. Chem. Int. Ed. Engl., Vol. 35, No. 13/14, 1996 and D. M. Bollag, Exp. Opin. Invest. Drugs, 6(7): 867-873, 1997).

Certain epothilones analogs having advantageous activity are represented by lactam compounds of formula I:

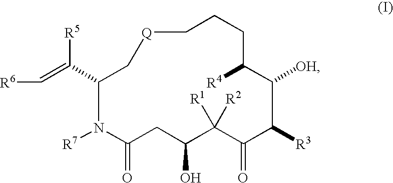

wherein the various symbols are as defined below. These compounds as well as other epothilone analogs are further described, for example, in U.S. Pat. Nos. 6,605,599; 6,262,094; 6,288,237; 6,613,912; and 6,831,076; each of which is assigned to the present assignee and incorporated herein by reference in its entirety. One particularly advantageous lactam compound of formula I is ixabepilone, which is described in example 3 of U.S. Pat. No. 6,605,599.

Before these lactam compounds can be used to treat diseases in patients, however, they must be formulated into pharmaceutical compositions that can be administered to the patients; for example, into a dosage form suitable for oral, mucosal (e.g., nasal, sublingual, vaginal, buccal, or rectal), parenteral (e.g., subcutaneous, intravenous, bolus injection, intramuscular, or intraarterial), or transdermal administration.

While the lactam compounds of formula I possess significant therapeutic properties, they also present challenges to those skilled in the art of pharmaceutical compounding as a result of certain chemical properties. These compounds, which contain a nitrogen moiety in the form of a lactam ring, generally have low water solubility and are difficult to formulate into aqueous media. Further, some of these compounds are susceptible to degradation in water. U.S. Pat. No. 6,670,384 discloses an intravenous composition prepared from a lactam compound of formula I. A lyophile was prepared by lyophilization of the lactam compound in a mixture of water and tertiary butanol. The intravenous composition was prepared by constituting the lactam compound lyophile in a mixture of Dehydrated Alcohol and a nonionic surfactant, such as a polyoxyethylated castor oil. However, the use of polyoxyethylated castor oil may present disadvantages such as, for example, potentially limiting the maximum dosage of a pharmaceutically active ingredient that is administered to a patient.

Desired in the art are compositions comprising the lactam compounds of formula I that may be administered parenterally at concentrations above their solubility values, preferably without administration of polyoxyethylated castor oil, and/or having enhanced stability.

In accordance with the present invention, a composition comprising nanoparticles of the lactam compounds of formula I is provided that is suitable for parenteral administration at higher concentrations than with existing formulations or alternatively, has sufficient stability to allow administration over longer periods of time than existing compositions such as those formulated with polyoxyethylated castor oil. Further, the composition of this invention may be formulated and administered to a patient in an aqueous carrier substantially free of organic solvent.

### SUMMARY OF THE INVENTION

The present invention relates to compositions comprising nanoparticles of at least one lactam compound of formula I. According to one aspect of the invention, the nanoparticles may be dispersed in a liquid medium. According to another aspect of the invention, at least one stabilizer may be adsorbed on the surfaces of the nanoparticles and can be present in an amount sufficient to provide the dispersed nanoparticles with an average particle diameter of less than about 1 micron. According to a different aspect of the invention, a solid composition is provided comprising the nanoparticles. Also provided is a method of treating cancer or other proliferative diseases comprising administering to a patient, a pharmaceutical composition comprising the nanoparticles.

### BRIEF DESCRIPTION OF THE DRAWING

FIG. 1. shows the plasma concentration of the lactam compound Ia, ixabepilone, as a function of time after bolus injection of the nanoparticle dispersions F1 to F4 and a control solution into rats.

### DETAILED DESCRIPTION OF THE INVENTION

### Definitions

The following are definitions of various terms used herein to describe the present invention. These definitions apply to the terms as they are used throughout this specification, unless otherwise limited in specific instances, either individually or as part of a larger group.

The term “alkyl” refers to optionally substituted straight- or branched-chain saturated hydrocarbon groups having from 1 to about 20 carbon atoms, preferably from 1 to about 7 carbon atoms. The expression “lower alkyl” refers to alkyl groups having from 1 to 4 carbon atoms. A “substituted lower alkyl” refers to an alkyl group having from 1 to 4 carbon atoms and one, two, or three (preferably one or two) substituents selected from those recited for “substituted alkyl” groups. The term “substituted alkyl” refers to an alkyl group substituted by, for example, one to four substituents (preferably one to two substituents), such as, halo, trifluoromethyl, trifluoromethoxy, hydroxy, alkoxy, cycloalkyoxy, heterocyclooxy, oxo (═O), alkanoyl, aryl, aryloxy, aralkyl, alkanoyloxy, amino, alkylamino, arylamino, aralkylamino, cycloalkylamino, heterocycloamino, disubstituted amino (in which the two substituents on the amino group are selected from alkyl, aryl, and aralkyl), alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido (e.g., SO₂NH₂), substituted sulfonamido, nitro, cyano, carboxy, carbamyl (e.g., CONH₂), substituted carbamyl (e.g., CONRR′, wherein R and R′ are selected from hydrogen, alkyl, and aryl, provided at least one of R and R′ is other than hydrogen), alkoxycarbonyl, aryl, substituted aryl, guanidino, and heterocyclo, such as indolyl, imidazolyl, furyl, thienyl, thiazolyl, pyrrolidyl, pyridyl, pyrimidyl, and the like. Wherein, as noted above, the substituents themselves are further substituted, such further substituents are selected from the group consisting of halogen, alkyl, alkoxy, aryl, and aralkyl. The definitions given herein for alkyl and substituted alkyl apply as well to the alkyl portion of alkoxy groups.

The term “halogen” or “halo” refers to fluorine, chlorine, bromine, and iodine.

The term “aryl” refers to an optionally substituted monocyclic or bicyclic aromatic hydrocarbon group having from about 6 to about 12 carbon atoms in the ring portion, for example, phenyl and naphthyl.

The term “aralkyl” refers to an aryl group bonded to a larger entity through an alkyl group, for example, a benzyl group.

The term “substituted aryl” refers to an aryl group substituted by, for example, one to four substituents (preferably one to two substituents) such as alkyl, substituted alkyl, halo, trifluoromethyl, trifluoromethoxy, hydroxy, alkoxy, cycloalkyloxy, heterocyclooxy, alkanoyl, alkanoyloxy, amino, alkylamino, dialkylamino, aralkylamino, cycloalkylamino, heterocycloamino, alkanoylamino, thiol, alkylthio, cycloalkylthio, heterocyclothio, ureido, nitro, cyano, carboxy, carboxyalkyl, carbamyl, alkoxycarbonyl, alkylthiono, arylthiono, alkysulfonyl, sulfonamido, aryloxy, and the like. The substituent may be further substituted by one or more members selected from the group consisting of halo, hydroxy, alkyl, alkoxy, aryl, substituted alkyl, substituted aryl, and aralkyl.

The term “cycloalkyl” refers to optionally substituted saturated cyclic hydrocarbon ring systems, preferably containing 1 to 3 rings and 3 to 7 carbons per ring, which may be further fused with an unsaturated C₃-C₇ carbocyclic ring. Exemplary groups include cyclopropyl, cyclobutyl, cyclopentyl, cyclohexyl, cycloheptyl, cyclooctyl, cyclodecyl, cyclododecyl, and adamantyl. Exemplary substituents include one or more alkyl or substituted alkyl groups as described above, or one or more of the groups described above as substituents for alkyl groups. Additionally, a cycloalkyl may contain a carbon-carbon bridge of one to two bridgehead carbon atoms, and/or one or two (preferably one) of the ring carbon atoms optionally may be replaced with a carbonyl group (substituted with keto).

The terms “heterocycle”, “heterocyclic” and “heterocyclo” refer to an optionally substituted, unsaturated, partially saturated, or fully saturated, aromatic or nonaromatic cyclic group, for example, which is a 4 to 7 membered monocyclic, 7 to 11 membered bicyclic, or 10 to 15 membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms, and sulfur atoms, where the nitrogen and sulfur heteroatoms may also optionally be oxidized and the nitrogen heteroatoms may also optionally be quaternized. The heterocyclic group may be attached at any heteroatom or carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolidinyl, 2-oxazepinyl, azepinyl, 4-piperidonyl, pyridyl, N-oxo-pyridyl, pyrazinyl, pyrimidinyl, pyridazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolane, and tetrahydro-1,1-dioxothienyl, dioxanyl, isothiazolidinyl, thietanyl, thiiranyl, triazinyl, triazolyl, and the like.

Exemplary bicyclic heterocyclic groups include benzothiazolyl, benzoxazolyl, benzothienyl, quinuclidinyl, quinolinyl, quinolinyl-N-oxide, tetrahydroisoquinolinyl, isoquinolinyl, benzimidazolyl, benzopyranyl, indolyl, indolizinyl, benzofuryl, chromonyl, coumarinyl, cinnolinyl, quinoxalinyl, indazolyl, pyrrolopyridyl, furopyridinyl (such as furo[2,3-c]pyridinyl, furo[3,1-b]pyridinyl], or furo[2,3-b]pyridinyl), dihydroisoindolyl, dihydroquinazolinyl (such as 3,4-dihydro-4-oxo-quinazolinyl), benzisothiazolyl, benzisoxazolyl, benzodiazinyl, benzofurazanyl, benzothiopyranyl, benzotriazolyl, benzpyrazolyl, dihydrobenzofuryl, dihydrobenzothienyl, dihydrobenzothiopyranyl, dihydrobenzothiopyranyl sulfone, dihydrobenzopyranyl, indolinyl, isochromanyl, isoindolinyl, naphthyridinyl, phthalazinyl, piperonyl, purinyl, pyridopyridyl, quinazolinyl, tetrahydroquinolinyl, thienofuryl, thienopyridyl, thienothienyl, and the like.

Smaller heterocyclos, such as, epoxides, aziridines, and the like, are also included.

Exemplary substituents for the groups “heterocycle,” “heterocyclic,” and “heterocyclo” include alkyl, substituted alkyl, or one or more substituent groups as described above for substituted alkyl or substituted aryl groups.

The term “alkanoyl” refers to —C(O)-alkyl.

The term “substituted alkanoyl” refers to —C(O)-substituted alkyl.

The term “heteroatoms” shall include oxygen, sulfur, and nitrogen.

The terms “diluent” and “infusion fluid” are used interchangeably herein to denote the fluid for administration to a patient, such as via parenteral (e.g., subcutaneous, intravenous, bolus injection, intramuscular, or intraarterial) administration.

The lactam compounds of formula I may form salts with a variety of organic and inorganic acids. Such salts include those formed with hydrogen chloride, hydrogen bromide, methanesulfonic acid, hydroxyethanesulfonic acid, sulfuric acid, acetic acid, trifluoroacetic acid, maleic acid, benzenesulfonic acid, toluenesulfonic acid, and various others as are recognized by those of ordinary skill in the art of pharmaceutical compounding. Such salts are formed by reacting a lactam compound of formula I in at least one equivalent amount of the acid in a medium in which the salt precipitates or in an aqueous medium followed by evaporation. In addition, zwitterions can be formed and are included within the term salts as used herein.

As used herein, the term “epothilone” refers to a compound selected from epothilone A, epothilone B, epothilone C, epothilone D, epothilone E, and epothilone F.

As used herein, the terms “lactam compound” and “lactam compound of formula I” refer to:

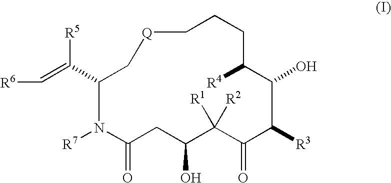

wherein Q is

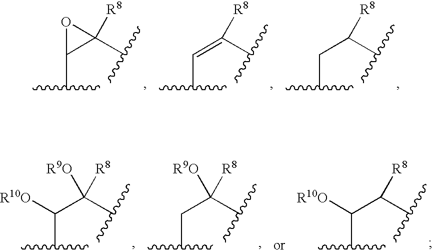

R¹, R², R³, R⁴, R⁵, R⁸, R⁹, and R¹⁰ are independently H, alkyl, substituted alkyl, and/or aryl, and when R¹ and R² are alkyl, they can be joined to form a cycloalkyl; and R⁶ and R⁷ are independently H, alkyl, substituted alkyl, cycloalkyl, aryl, and/or heterocyclo.

In one limiting embodiment, the lactam compound is selected from lactam compounds of formula I wherein Q is

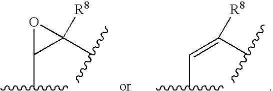

In a different limiting embodiment, the lactam compound is selected from lactam compounds of formula I wherein R⁶ is heterocyclo, such as thiazolyl or substituted thiazolyl.

A preferred lactam compound of formula I is:

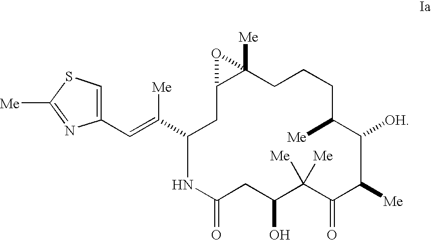

The lactam compound of formula Ia, also referred to as “lactam compound Ia”, is ixabepilone, which has the chemical name:
[1S-[1R*,3R*(E),7R*,10S*,11R*,12R*,16S*]]-7,11-Dihydroxy-8,8,10,12,16-pentamethyl-3-[1-methyl-2-(2-methyl-4-thiazolyl)ethenyl]-4-aza-17-oxabicyclo[14.1.0]heptadecane-5,9-dione.

Methods of preparing the lactam compound of formula Ia, ixabepilone, are described in U.S. Pat. No. 6,518,421, and U.S Patent Application Publication 2004/0132146A1, the disclosures of which are incorporated herein by reference.

The D₅₀ value refers to a parameter for a population of particles in which 50% of the particles have diameters less than the D₅₀ value and 50% of the particles have diameters greater than the D₅₀ value, based on volume distribution. The D₁₀₀ value refers to a parameter representing the minimum value at which 100% of the particles have diameters less than the D₁₀₀ value. The D₅₀ and the D₁₀₀ values may be determined by a suitable static laser light scattering technique, such as measurement by the Horiba™ LA-910 Laser Diffraction Particle Size Analyzer (Horiba, Ltd., Japan). As used herein “average particle diameter” refers to the D₅₀ value. Optical microscopy may be employed to verify the absence of large agglomerates.

The compositions of this invention comprise nanoparticles of the lactam compound of formula I. The nanoparticles may have an average particle diameter of less than about 1 micron, preferably less than about 700 nanometers (nm), and more preferably less than about 500 nm. Preferred ranges for the average particle diameter of the nanoparticles may include a range of from about 50 nm to about 1 micron, preferably a range of from about 100 nm to about 700 nm, and more preferably a range of from about 100 nm to about 500 nm. In one embodiment, the nanoparticles of the lactam compound of formula I also have a D₁₀₀ value of less than about 5 microns, preferably less than about 4 microns, and most preferably, less than about 2.5 microns. The nanoparticle dispersion of this invention may be filtered through a 5 micron pore filter prior to particle size determination.

The lactam compound may be present in the nanoparticles as crystals, in amorphous form, or a mixture thereof. Crystals are preferred. The compositions of this invention may contain one or more different polymorphs of the lactam compound of formula I. Crystalline polymorphs of the lactam compound of formula Ia are described in U.S. Pat. No. 6,689,802, the disclosure of which is incorporated herein by reference.

Crystals of the lactam compound of formula I can be prepared by methods known in the art, such as those described in WO 00/39276, WO 02/14323, WO 03/070170, Crystallization Processes, Ohtaki, H., Wiley (1998), and Handbook of Industrial Crystallization, Meyerson, Allan S., Butterworth-Heinemann (1993). Suitable techniques to characterize these crystals are know in the art and include powder x-ray diffraction techniques. For example, a solution of the lactam compound in a suitable solvent, such as ethyl acetate, isobutyl acetate, n-butyl acetate, toluene, isopropyl acetate, methyl tertiary butyl ether, or methyl isobutyl ketone as a single solvent or in combination with antisolvents, such as hexane, n-heptane, or cyclohexane at ambient temperature or at a temperature up to the boiling temperature is prepared or obtained from a process stream. The solution may be supersaturated by adding a suitable antisolvent or by lowering the temperature, or a combination of both with or without agitation. An extended period of heating at or below the boiling temperature of the mixture is optionally employed to control crystal characteristics. The resulting crystals may be collected by filtration and dried at normal atmospheric pressure or at reduced pressure, with the optional application of heat.

The nanoparticle dispersion of this invention comprises nanoparticles of the lactam compound of formula I, at least one stabilizer, and a liquid medium. The nanoparticle dispersion may contain a sufficient concentration of nanoparticles to allow administration of an effective amount of the nanoparticles to a patient in need thereof; and yet not too great a concentration of nanoparticles such that the nanoparticle dispersion is too viscous or unstable. For example, the nanoparticle dispersion may comprise in the range of from about 0.1 to about 40 weight %, preferably in the range of from about 0.5 to about 20 weight %, and more preferably in the range of from about 1 to about 10 weight % of the nanoparticles, based on the weight of the nanoparticle dispersion.

In one embodiment, the nanoparticle dispersion comprises two or more different types of lactam compounds of formula I. In a preferred embodiment, the nanoparticle dispersion comprises the lactam compound of formula Ia.

The nanoparticle dispersion may also contain at least one stabilizer. The stabilizer may be adsorbed on the surfaces of the nanoparticles. Typically, the nanoparticles are dispersed into a liquid medium, preferably in the presence of the stabilizer. The stabilizer may be employed as an adjuvant to aid in the wetting and/or the separation of the individual nanoparticles during the dispersion process. The ability of a stabilizer to aid in the wetting and/or the separation of the individual nanoparticles may be determined by comparing the nanoparticle dispersion processes for a composition containing the stabilizer and a control composition without the stabilizer. The ability of a stabilizer to aid in the wetting and/or separation of individual nanoparticles may be indicated by shorter dispersion times to obtain nanoparticle dispersions of the same average particle diameter, or smaller average particles diameters for the same dispersion time, under similar processing conditions. Alternatively, the stabilizer may be employed to promote stability of the dispersed nanoparticles in the liquid medium, preferably an aqueous medium. The ability of a stabilizer to promote the stability of the nanoparticles may be determined by less settling of the nanoparticles after a period of 24 hours at 20° C. for the nanoparticle dispersion comprising the stabilizer compared to a control nanoparticle dispersion without the stabilizer. Alternatively, stability can be ascertained by an increase of less than 200 nm, preferably less than 50 nm, in the D₅₀ value as determined by static laser light scattering. Further, the stability may also be ascertained by the absence or near absence of agglomerates or particles greater than 5 microns, preferably greater than 1 micron.

As used herein, “adsorbed on the surface” indicates that the stabilizer is associated with the surface of the nanoparticles, but only to such a degree or extent that the stabilizer does not materially interfere with bioavailability of the lactam compound. For example, the stabilizer may be physically adsorbed to the surface of the particle. Alternatively, the stabilizer may be bonded to the surface, such as, for example, by covalent bonds, hydrogen bonds, or van der Waals bonds. Two or more stabilizers may be employed to optimize the dispersion of the nanoparticles into the liquid medium and/or the stability of the dispersed nanoparticles. For example, a first stabilizer may be employed to aid in the wetting and the separation of the individual nanoparticles, and a second stabilizer may be employed to provide stability to the dispersed nanoparticles in the liquid medium.

The stabilizer may be selected from organic and/or inorganic pharmaceutical excipients and/or other substances that aid in the wetting or stabilization of the nanoparticles. Suitable stabilizers include, for example, various polymers, low molecular weight oligomers, natural products, enzymes, and/or surfactants, such as nonionic and anionic surfactants. Examples of suitable stabilizers may include, but are not limited to, gelatin, casein, lecithin, gum acacia, cholesterol, tragacanth, stearic acid, benzalkonium chloride, calcium stearate, sodium deoxycholate, cholic acid, bile salts, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, phosphates, lysozyme, sodium dodecylsulfate, carboxymethylcellulose calcium, carboxymethylcellulose sodium, methylcellulose, hydroxyethylcellulose, albumin, hydroxypropylcellulose, hydroypropylmethylcellulose phthalate, noncrystalline cellulose, magnesium aluminum silicate, polyvinyl alcohol, polyvinylpyrrolidone such as grades K29/32, K30, K13-19, K12, and K17, copolymers of vinyl acetate and vinylpyrrolidone, and polyethylene glycol-derivatized lipids such as those disclosed in U.S. Pat. No. 6,270,806, which is incorporated herein. Examples of polyethylene glycol-derivatized lipids include polyethylene glycol-phospholipids, polyethylene glycol-cholesterol, polyethylene glycol-cholesterol derivative, polyethylene glycol-vitamin A, and polyethylene glycol-vitamin E. Examples of suitable nonionic block copolymers include polaxamers such as block copolymers of ethylene oxide and propylene oxide. Examples of polaxamers include, but are not limited to, Pluronic™ F68 copolymer and Pluronic™ F108 copolymer (BASF Corporation).

Generally, the nanoparticle dispersion may contain one or more stabilizers in the range of from about 0.01 to about 10 weight %, preferably in the range of from about 0.2 to about 7 weight %, and more preferably from about 0.5 to about 4 weight %, based on the weight of the nanoparticle dispersion.

The nanoparticle dispersion also comprises a liquid medium in which the nanoparticles are dispersed. The liquid medium may comprise one or more solvents, such as, for example, organic solvents and/or water. The one or more lactam compounds have sufficiently low solubility in the liquid medium such that the one or more lactam compounds do not fully solubilize but form particles in the liquid medium.

In a preferred embodiment, the liquid medium is an aqueous medium that comprises a predominant amount of water and optionally, a minor amount of one or more water miscible organic solvents. A minor amount of the optional water miscible organic solvents is less than 50 weight %, preferably less than 25 weight %, and more preferably less than 10 weight %, based on the weight of the aqueous medium. Examples of suitable optional water miscible organic solvents include ethanol, propylene glycol, polyethylene glycol, dimethyl acetamide, glycerol, isopropanol, acetone, dimethyl formamide, methylene chloride, and tertiary butyl alcohol. Generally, the pH of the nanoparticle dispersion comprising the aqueous medium is in a range that allows for the parenteral administration of the nanoparticle dispersion to a mammal. Suitable pH values for the nanoparticle dispersion of this embodiment include, but are not limited to, a pH in the range of from about 2 to about 10, preferably in the range of from about 4 to about 9, and more preferably in the range of from 6 to about 8. One standard method to determine the pH of the nanoparticle dispersion of this embodiment is measurement with a combination electrode at a temperature of about 25° C.

In one limiting embodiment, the nanoparticle dispersion comprises an aqueous medium that is substantially free of organic solvent. As used herein, “substantially free of organic solvent” means containing less than about 10 weight %, preferably less than about 5 weight %, and more preferably less than about 1 weight % organic solvent, based on the weight of the nanoparticle dispersion. In one further limiting embodiment, suitable ranges of organic solvent include from zero to about 10 weight %, preferably from zero to about 5 weight %, and more preferably from zero to about 1 weight %. Most preferred is a nanoparticle dispersion that comprises about zero weight % organic solvent, and most preferably, zero weight % organic solvent.

The stability of the lactam compound of formula I in the nanoparticle dispersion of this invention may be determined by measuring the rates of degradation of the lactam compound of formula I in the nanoparticle dispersion at a concentration of about 1 mg/ml and in a solution having a concentration of 0.1 mg/ml, wherein the nanoparticle dispersion and solution have the same liquid medium. The rate of degradation is measured over a 24 hour period while maintaining the nanoparticle dispersion and the solution at the same conditions, such as temperature, pH, and same light exposure. Improved stability of the lactam compound is indicated by a lower rate of degradation of the lactam compound in the nanoparticle dispersion as compared with the lactam compound in the solution. The degradation of the lactam compound may be determined by measuring the level of impurities. Preferably, the degradation rate for the lactam compound in the nanoparticle dispersion is less than half of the degradation rate of the lactam compound in solution, more preferably, the degradation rate for the lactam compound in the nanoparticle dispersion is less than one quarter of the degradation rate of the lactam compound in solution, and most preferably, the degradation rate for the lactam compound in the nanoparticle dispersion is less than one tenth of the degradation rate of the lactam compound in solution.

The nanoparticle dispersion may be prepared by various methods, including methods that provide impact, shear, or cavitation forces such as homogenization, sonication, grinding, counter current flow homogenization, or microfluidization; or precipitation. A combination of methods may be employed. Dry nanoparticles may be prepared by spray methods employing a nozzle or capillary such as supercritical fluid methods, cryogenic methods, or spray drying. The dry nanoparticles may be dispersed into the liquid medium in the presence of at least one stabilizer to provide the nanoparticle dispersion.

In one method, the nanoparticle dispersion may be prepared by admixing particles of at least lactam compound in a liquid medium. The particles may be amorphous or crystalline material. A grinding step may be employed in which the admixture is subjected to grinding in the presence of grinding media to reduce the size of the particles to provide a nanoparticle dispersion having nanoparticles with an average particle diameter of less than about 1 micron. One or more stabilizers may be added before, during, and/or after the grinding step, or any combination thereof. In one embodiment, the stabilizer is added prior to grinding to aid in the separation and stabilization of the resulting nanoparticles. Various media mills may be employed including, for example, ball mills, attritor mills, vibratory mills, and media mills such as bead mills and sand mills. Suitable media for grinding media include particles of metal oxides such as zirconium oxide; zirconium silicate; ferrite; stainless steel; titania; alumina; glass; and polymeric beads, such as polystyrene crosslinked with divinylbenzene, styrene copolymers, polycarbonates, polyacetals, vinyl chloride polymers and copolymer, polyurethanes, polyamides, polytetrafluorethylenes, polyhydroxymethacrylate, polyhydroxyethylacrylate, and silicone containing polymers.

In another method, the nanoparticle dispersion is prepared by a precipitation technique. In this method, the lactam compound is dissolved in a suitable solvent; admixed with a second solution comprising one or more stabilizers; and then precipitated using an appropriate anti-solvent to obtain nanoparticles having an average particle diameter of less than about 1 micron. If not already in the aqueous medium, these nanoparticles may be dispersed into the aqueous medium, optionally with mixing at low or high shear, to provide the nanoparticle dispersion of this invention.

Techniques to provide pharmaceutically active ingredients as nanoparticle dispersions having average particle sizes of less than 1 micron are disclosed in U.S. Pat. No. 5,145,684; U.S. Pat. No. 5,833,891; U.S. Pat. No. 6,113,795; U.S. Pat. No. 6,264,922 B1; U.S. Pat. No. 6,270,806 B1; U.S. Pat. No. 6,555,139 B1; WO 02/094215 A2; WO 03/049718 A1, and E. Merisko-Liversidge et al., European J. Pharmaceutical Sci., 18, 113-120 (2003), each of which is incorporated herein by reference.

In one limiting embodiment, a nanoparticle dispersion of this invention is prepared in an aqueous medium or nonaqueous medium, and then dried to provide lyophilized material. The lyophilized material comprises nanoparticles of at least one lactam compound of formula I. Other suitable drying methods include evaporation, spray drying, spray or wet granulation, and spray-coating. The nanoparticle dispersion may be prepared by dispersing the dried material into the liquid medium, typically with mixing or sonication.

Optionally, the nanoparticle dispersion comprises excipients or other materials including preservatives, such as methyl paraben, ethyl paraben, propyl paraben, benzyl alcohol, and thiomersal; pH modifiers or buffers such as sodium hydroxide, hydrochloric acid, phosphates, citrates, tris(hydroxymethyl)aminomethane, and borates; bulking agents or cryoprotectants such as mannitol, dextran, dextrose, sodium chloride, trehalose, sucrose, tyloxapol, and amino acids; viscosity modifying agents such as methyl cellulose, sodium carboxymethyl cellulose, gelatin, microcrystalline cellulose, Polyox™ water soluble resin (Dow Chemical Co., MI), vitamin E TPGS (D-alpha tocopheryl polyethylene glycol succinate), polyethylene glycols, propylene glycols, and glycerin; antioxidants such as ascorbic acid or its salts; metal chelating agents such as ethylene diamine tetraacetate and its salts; and cyclodextrins or cyclodextrin derivatives.

In another embodiment, the nanoparticle dispersion is a pharmaceutical composition suitable for parenteral administration to a mammal. Examples of parenteral administration includes subcutaneous, intravenous, intramuscular, and intraarterial administration; and bolus injection. Preferred is intravenous administration. The pharmaceutical composition of this embodiment may comprise nanoparticles of at least one lactam compound, at least one stabilizer, and the aqueous medium; wherein the nanoparticles are dispersed in the aqueous medium, and the at least one stabilizer is adsorbed on surfaces of the nanoparticles in an amount sufficient to provide the nanoparticles with an average particle diameter of less than about 1 micron. Preferably, the pharmaceutical composition of this embodiment comprises nanoparticles having an average particle diameter of less than about 600 nm, and more preferably, less than about 500 nm. The pharmaceutical composition of this embodiment optionally contains excipients or other materials; for example, suitable non-toxic, parentally acceptable diluents or solvents, such as mannitol, 1,3-butanediol, Lactated Ringer's Injection, or an isotonic sodium chloride solution. Preferably, the pharmaceutical composition of this embodiment is substantially free of organic solvent. Preferably, the pharmaceutical composition of this embodiment has a pH in the range of from about 6 to about 8. Preferably, the pharmaceutical composition of this embodiment contain less than about 5 weight % of polyoxyethylated castor oil surfactant, and more preferably has about zero weight % polyoxyethylated castor oil surfactant, and most preferably zero weight % polyoxyethylated castor oil surfactant.

The nanoparticle dispersion may be sterilized by suitable sterilization techniques, which may be employed prior to, during, and/or after the preparation of the nanoparticle dispersion. Suitable techniques include the application of heat, exposure to radiation, chemical treatment, filtration, or a combination thereof. Techniques employing autoclaving, which are suitable for sterilizing nanoparticle compositions, are disclosed in U.S. Pat. Nos. 5,298,262, 5,346,702, 5,352,459, and 5,534,270.

In one aspect of the present invention, a solid nanoparticulate composition is provided comprising nanoparticles of the lactam compound of formula I, wherein the nanoparticles have an average diameter of less than about 1 micron. Preferably, the solid nanoparticulate composition comprises nanoparticles having an average particle diameter of less than about 700 nm, and more preferably, less than about 500 nm. The solid nanoparticulate composition may comprise two or more different types of lactam compounds of formula I; for example, a mixture of nanoparticles of two different lactam compounds of formula I. In a preferred embodiment, the solid nanoparticulate composition comprises the lactam compound of formula Ia. The solid nanoparticulate composition has a solid, dry form, such as a dry powder. Low levels or trace amounts of water and/or solvent may be present in the solid nanoparticulate composition. Preferably, the solid nanoparticulate composition comprises less than 6 weight %, more preferably, less than 2 weight %, and most preferably, less than 1 weight % water and/or solvent, based on the weight of the solid nanoparticulate composition.

In one embodiment, the solid nanoparticulate composition may optionally comprise at least one stabilizer. The stabilizer may be absorbed on the surfaces of the nanoparticles. The stabilizer may be used in the preparation of the solid nanoparticulate composition, for example, in the preparation of the nanoparticle dispersion of this invention followed by subsequent drying; and/or may aid in the redispersion of the dry nanoparticles to constitute the nanoparticle dispersion. The removal of the liquid medium from the nanoparticle dispersion provides a solid nanoparticulate composition with the same ratio of stabilizer to nanoparticles as present in the nanoparticle dispersion. Optionally, additional stabilizer may be added to the solid nanoparticulate composition.

The solid nanoparticulate composition may optionally comprise at least one pharmaceutically acceptable excipient. Examples of suitable excipients include diluents such as lactose, microcrystalline cellulose, dextrin, dextrose, mannitol, and xylitol; binders such as starch, hydroxypropylmethylcellulose, povidone, and hydroxypropylcellulose; disintegrants such as crospovidone, croscarmellose, sodium alginate, and pregelatinized starch; glidants such as talc and colloidal silicon dioxide; lubricants such as magnesium stearate, stearic acid, polyethylene glycol, and sodium stearyl fumarate; and wetting agents such as docusate sodium, sodium lauryl sulfate, and polysorbates such as, for example, Tween™ 80 surfactant (ICI Americas Inc., NJ). The solid nanoparticulate composition may be formulated according to methods known in the art, as found in Remington's Pharmaceutical Science, Gennaro, Alfonso R., Remington, Joseph P., Mack Publishing Co., Easton, Pa. (1995). The solid nanoparticulate composition may be administered orally, bucally, or sublingually as tablets, capsules, granules, powders such as, for example, lyophiles, or coated beads. Further, the solid nanoparticulate composition may be administered in a form suitable for immediate release or extended release.

### Utility

The lactam compounds of formula I are useful as microtubule-stabilizing agents. They are useful in the treatment of a variety of cancers and other proliferative diseases including, but not limited to, the following;

carcinoma, including that of the bladder, breast, colon, kidney, liver, lung, ovary, pancreas, stomach, cervix, thyroid, spleen, prostate, and skin, including squamous cell carcinoma;

hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, and Burkitts lymphoma;

hematopoietic tumors of myeloid lineage, including acute and chronic myelogenous leukemias and promyelocytic leukemia;

tumors of the central and peripheral nervous system, including astrocytoma, neuroblastoma, glioma, and schwannomas;

tumors of mesenchymal origin, including fibrosarcoma, rhabdomyosarcoma, and osteosarcoma; and

other tumors, including melanoma, xeroderma pigmentosum, seminoma, keratoacanthoma, thyroid follicular cancer, and teratocarcinoma.

The lactam compounds of formula I are useful for treating patients who have been previously treated for cancer, as well as those who have not previously been treated for cancer. The methods and compositions of this invention can be used in first-line and second-line cancer treatments. Furthermore, the lactam compounds of formula I are useful for treating refractory or resistant cancers. The lactam compounds of formula I will also inhibit angiogenesis, thereby affecting the growth of tumors and providing treatment of tumors and tumor-related disorders. Such anti-angiogenesis properties will also be useful in the treatment of other conditions responsive to anti-angiogenesis agents including, but not limited to, certain forms of blindness related to retinal vascularization, arthritis, especially inflammatory arthritis, multiple sclerosis, restinosis, and psoriasis.

The lactam compounds of formula I will induce or inhibit apoptosis, a physiological cell death process critical for normal development and homeostasis. Alterations of apoptotic pathways contribute to the pathogenesis of a variety of human diseases. The subject compounds, as modulators of apoptosis, will be useful in the treatment of a variety of human diseases with aberrations in apoptosis including, but not limited to, cancer and precancerous lesions, immune response related diseases, viral infections, kidney disease, and degenerative diseases of the musculoskeletal system.

The lactam compounds of formula I may also be formulated or co-administered with other therapeutic agents that are selected for their particular usefulness in administering therapies associated with the aforementioned conditions. The lactam of compounds of formula I may be formulated with agents to prevent nausea, hypersensitivity, and gastric irritation, such as anti-emetics, and H₁ and H₂ antihistamines. The above therapeutic agents, when employed in combination with a compound of formula I, may be used in those amounts indicated in the Physicians' Desk Reference (PDR) or as otherwise determined by one of ordinary skill in the art.

Furthermore, the lactam compounds of formula I may be administered in combination with other anti-cancer and cytotoxic agents and treatments useful in the treatment of cancer or other proliferative diseases. Especially useful are anti-cancer and cytotoxic drug combinations wherein the second drug chosen acts in a different manner or different phase of the cell cycle, e.g., S phase, than the present compounds of formula I which exert their effects at the G₂-M phase. Examples of classes of anti-cancer and cytotoxic agents include, but are not limited to, alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (TAXOL®), docetaxel (TAXOTERE®); plant-derived products, such as vinca alkaloids, epipodophyllotoxins, and taxanes; topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors, immune modulators, and monoclonal antibodies. The lactam compounds of formula I may also be used in conjunction with radiation therapy.

Representative examples of these classes of anti-cancer and cytotoxic agents include, but are not limited to, mechlorethamine hydrochloride, cyclophosphamide, chlorambucil, melphalan, ifosfamide, busulfan, carmustin, lomustine, semustine, streptozocin, thiotepa, dacarbazine, methotrexate, thioguanine, mercaptopurine, fludarabine, pentastatin, cladribin, cytarabine, fluorouracil, doxorubicin (including salts such as doxorubicin hydrochloride), daunorubicin, idarubicin, bleomycin sulfate, mitomycin C, actinomycin D, safracins, saframycins, quinocarcins, discodermolides, vincristine, vinblastine, vinorelbine tartrate, etoposide (including salts such as etoposide phosphate), teniposide, paclitaxel, tamoxifen, estramustine, estramustine phosphate sodium, flutamide, buserelin, leuprolide, pteridines, diyneses, levamisole, aflacon, interferon, interleukins, aldesleukin, filgrastim, sargramostim, rituximab, BCG, tretinoin, irinotecan hydrochloride, betamethosone, capecitabine, gemcitabine hydrochloride, altretamine, and topoteca and analogs or derivatives thereof.

Other examples of these classes of anticancer and cytotoxic agents include, but are not limited to, cisplatin, carboplatin, caminomycin, aminopterin, methotrexate, methopterin, ecteinascidin 743, porfiromycin, 5-fluorouracil (5-FU), 6-mercaptopurine, gemcitabine, cytosine arabinoside, paclitaxel, doxorubicin, daunorubicin, mitomycin C, podophyllotoxin or podophyllotoxin derivatives such as etoposide, etoposide phosphate or teniposide, melphalan, vinblastine, vincristine, leurosidine, vindesine, and leurosine. It is to be understood that the compounds of formula I may be administered in combination with particular anticancer and cytotoxic agents falling within these classes of agents, for example, the compounds of formula I may be administered in combination with any 5-FU agents, and/or prodrugs thereof, including without limitation capecitabine (XELODA®).

Further examples of anti-cancer and other cytotoxic agents include the following: cyclin dependent kinase inhibitors as found in WO 99/24416; and prenyl-protein transferase inhibitors as found in WO 97/30992 and WO 98/54966.

Without wishing to be bound to any mechanism or morphology, it is expected that the lactam compounds of formula I could also be used to treat conditions other than cancer or other proliferative diseases. Such conditions include, but are not limited to viral infections such as herpesvirus, poxvirus, Epstein-Barr virus, Sindbis virus, and adenovirus; autoimmune diseases such as systemic lupus erythematosus, immune mediated glomerulonephritis, rheumatoid arthritis, psoriasis, inflammatory bowel diseases, and autoimmune diabetes mellitus; neurodegenerative disorders such as Alzheimer's disease, AIDS-related dementia, Parkinson's disease, amyotrophic lateral sclerosis, retinitis pigmentosa, spinal muscular atrophy, and cerebellar degeneration; AIDS; myelodysplastic syndromes; aplastic anemia; ischemic injury associated myocardial infarctions; stroke and reperfusion injury; restenosis; arrhythmia; atherosclerosis; toxin-induced or alcohol induced liver diseases; hematological diseases such as chronic anemia and aplastic anemia; degenerative diseases of the musculoskeletal system such as osteoporosis and arthritis; aspirin-sensitive rhinosinusitis; cystic fibrosis; multiple sclerosis; kidney diseases; and cancer pain.

The effective amount of one of the lactam compounds of formula I may be determined by one of ordinary skill in the art, and includes exemplary dosage amounts for a human for treatment of cancer or other proliferative diseases of from about 0.01 to 200 mg/kg/day, which may be administered in a single dose or in the form of individual divided doses, such as from 1 to 4 times per day. Preferably, the subject compounds are administered in a dosage of less than 100 mg/kg/day, in a single dose or in 2 to 4 divided doses. The lactam compound of formula I may be administered by infusion with infusion times of less than about 24 hours, for example, infusion times of 1 to 3 hours. For example, metastatic breast cancer may be treated by administering a dose of up to 50 mg/m² of the lactam compound of formula I once per day every 21 days, given in a total volume of about 200 mL, and with an infusion time of less than 24 hours. In another example, metastatic breast cancer may be treated by administering the lactam compound of formula I at a dose of up to 20 mg/m² once per day every week. In a further example, metastatic breast cancer may be treated by administering the lactam compound of formula I at a dose of about 6 to 8 mg/m² daily for five consecutive days in a treatment cycle of 21 days. The treatment cycle may be repeated one or more times as needed. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. The nanoparticle dispersion of this invention may be administered parenterally; however, other routes of administration are contemplated herein as are recognized by those skilled in the oncology arts.

The concentration of the lactam compound of formula I in the nanoparticle dispersion and the length of time for parenterally administering the nanoparticle dispersion may be varied to obtain a desired level of the lactam compound of formula I in the patient. For example, the nanoparticle dispersion of this invention may be administered in a short period of time, such as 10 to 30 minutes, by parenteral administration, such as, for example, by intravenous administration (IV), to provide nanoparticles of the lactam compound of formula I to the patient. After administration, the nanoparticles dissolve within the patient to provide an effective dose of the lactam compound of formula I. Alternatively, the nanoparticle dispersion may be diluted immediately prior to administration with an infusion fluid or diluent to provide an intravenous solution containing the lactam compound of formula I. This intravenous solution may be administered for a period of up to 24 hrs.

Preferred subjects for treatment include animals, most preferably mammalian species such as humans, and domestic animals such as dogs, cats and the like, subject to the aforementioned disorders.

The nanoparticle dispersion of this invention is suitable as a pharmaceutical composition suitable for treating cancer or other proliferative diseases. The pharmaceutical composition may have a concentration of about 1 mg/mL to about 50 mg/mL of the one or more nanoparticles of the lactam compound of formula I. The solid nanoparticulate composition may be provided as a lyophile for constitution, for example, packaged in quantities of 10 to 100 mg/vial, preferably 20 to 80 mg/vial, and most preferably 40 to 60 mg/vial of the lactam compound as nanoparticles. The lyophile may be constituted to provide a drug concentration in the range of 1 to 50 mg/mL, preferably in the range of from 1 to 10 mg/mL, prior to administration. In one limiting embodiment, a lyophilized composition comprising the nanoparticles of the lactam compound and the at least one stabilizer is combined with the aqueous medium to reconstitute the nanoparticle dispersion. This nanoparticle dispersion may be further diluted with one or more suitable parenteral diluents to provide a composition suitable for parenteral administration. Such diluents are well known to those of ordinary skill in the art. These diluents are generally available in clinical facilities. Suitable diluents include 5% Dextrose Injection, Lactated Ringer's Injection, Sterile Water for Injection, and the like. A preferred diluent is Lactated Ringer's Injection. Per 100 mL, Lactated Ringer's Injection contains Sodium Chloride USP 0.6 g, Sodium Lactate 0.31 g, Potassium chloride USP 0.03 g and Calcium Chloride.2H₂O USP 0.02 g. The osmolarity is 275 mOsmol/L, which is very close to isotonicity. The final concentration for administration would preferably contain from about 0.5 mg/mL to about 2.5 mg/mL of the one or more nanoparticles of the lactam compound. Preferably the pharmaceutical composition has a pH in the range of from 6 to 8.

Typically the lactam compounds of formula I are administered until the patient shows a response, for example, a reduction in tumor size, or until dose limiting toxicity is reached. One of ordinary skill in the art will readily know when a patient shows a response or when dose limiting toxicity is reached. The common dose limiting toxicities associated with the epothilone analogs include, but are not limited to, fatigue, arthralgia/myalgia, anorexia, hypersensitivity, neutropenia, thrombocytopenia, and neurotoxicity.

Generally, the nanoparticles of the lactam compounds of formula I are administered by IV infusion over a period of from about 10 minutes to about 24 hours. Examples of other suitable periods for infusion include, from about 30 minutes to about 3 hours, from about 45 minutes to about 2 hours, about 1 hour, and about 3 hours. Typically, the nanoparticles of the lactam compounds of formula I are administered intravenously in a dose of from about 0.5 mg/m² to about 100 mg/m² preferably about 1 mg/m² to about 80 mg/m², more preferably about 2.5 mg/m² to about 60 mg/m², and most preferably about 40 mg/m². One of ordinary skill in the art would readily know how to convert doses from mg/kg to mg/m² given either or both the height and or weight of the patient (See, e.g., http://www.fda.gov/cder/cancer/animalframe.htm).

As discussed above, the nanoparticles of the lactam compounds of formula I can be administered intravenously, orally, or both intravenously and orally. In particular, the methods of the invention encompass dosing protocols such as once a day for 2 to 10 days, preferably every 3 to 9 days, more preferably every 4 to 8 days and most preferably every 5 days. In one limiting embodiment there is a period of 3 days to 5 weeks, preferably 4 days to 4 weeks, more preferably 5 days to 3 weeks, and most preferably I week to 2 weeks, in between cycles where there is no treatment. In another limiting embodiment, the nanoparticles of the lactam compounds of formula I can be administered intravenously, or both intravenously and orally, once a day for 3 days, with a period of preferably 1 week to 3 weeks in between cycles where there is no treatment. In yet another limiting embodiment, the nanoparticles of the lactam compounds of formula I are administered once a day for 5 days, with a period of preferably 1 week to 3 weeks in between cycles where there is no treatment.

In one preferred limiting embodiment, the treatment cycle for intravenous administration of the nanoparticles of the lactam compounds of formula I is once daily for 5 consecutive days and the period between treatment cycles is from 2 to 10 days, preferably one week.

The nanoparticles of the lactam compounds of formula I can also be administered intravenously, or both intravenously and orally once every 1 to 10 weeks, preferably every 2 to 8 weeks, more preferably every 3 to 6 weeks, and even more preferably every 3 weeks.

In another method of the invention, the nanoparticles of the lactam compounds of formula I are administered in a 28 day cycle wherein the lactam compounds of formula I are intravenously administered on days 1, 7, and 14 and orally administered on day 21. Alternatively, the nanoparticles of the lactam compounds of formula I are administered in a 28 day cycle wherein the lactam compounds of formula I are orally administered on day 1 and the nanoparticles of the lactam compounds of formula I are intravenously administered on days 7, 14, and 28.

### EXAMPLES

The following examples are provided, without any intended limitation, to further illustrate the present invention.

Abbreviations:

| | |
|---|---|
| cc | cubic centimeter |
| EDTA | ethylenediaminetetraacetic acid |
| g | gram |
| kg | kilogram |
| kPa | kilopascal |
| mg | milligram |
| mL | milliliter |
| mm | millimeter |
| mt | millitorr |
| ng | nanogram |
| rpm | rotations per minute |
| wt. % | weight % |

In the examples, deionized water refers to water that was deionized and distilled to a resistance of greater than 18 Mohms.

Particle Size Measurements: The average particle size of the nanoparticle dispersion was measured using a Horiba™ LA-910 Laser Diffraction Particle Size Analyzer using a relative refractive index setting of 1.20-0.10i standard mode. Samples for analysis were prepared by diluting approximately 40-80 microliter of the nanoparticle dispersion in 9 mL of deionized water to provide 60-80% transmittance in the analyzer.

Nanoparticle dispersions were prepared comprising the lactam compound of formula Ia, [1S-[1R*,3R*(E),7R*,10S*,11R*,12R*,16S*]]-7,11-Dihydroxy-8, 8,10,12,16-pentamethyl-3-[1-methyl-2-(2-methyl-4-thiazolyl)ethenyl]-4-aza-17-oxabicyclo[14.1.0]heptadecane-5,9-dione according to the following procedures.

### Example 1

### Preparation of a Nanoparticle Dispersion Containing 5 wt. % Lactam Compound Ia and 2 wt. % Pluronic™ F108 Surfactant

A stock solution of 5 wt. % Pluronic™ F108 surfactant (BASF Corp.) was prepared in deionized water and filtered though a 0.2 micron filter. The following materials were introduced into the jacketed 10-mL milling chamber of a NanoMill™-01 mill (Elan Pharma International Limited, Ireland) in the order listed: 5.43 g of 0.5 mm polystyrene milling media, 0.233 g of the lactam compound of formula Ia, 1.865 mL of a filtered solution containing 5 wt. % Pluronic™ F108 surfactant, and 2.563 mL of deionized water. The mill was assembled, and the milling chamber was equilibrated to a temperature of 4-5° C. by means of a circulating thermostatted water bath. The contents were milled for 2 minutes at 1800 rpm, followed by 43 minutes at 5500 rpm. The resulting nanoparticle dispersion was separated from the polystyrene milling beads by aspirating into a 3-mL Sub-Q syringe fitted with a 26 gauge ⅝ inch needle, and transferred to a suitable vial. Yield=3.05 g (˜65 wt. %).

### Example 2

### Preparation of a Nanoparticle Dispersion Containing 10 wt. % Lactam Compound Ia, 1.8 wt. % Polyvinylpyrrolidone(PVP), and 0.2 wt. % Sodium Deoxycholate

Stock solutions of 4 wt. % PVP (˜10,000 MW) and 0.4 wt. % sodium deoxycholate were prepared in Water for Injection and filtered through a 0.45 micron filter. The following materials were introduced into the jacketed 50-mL milling chamber of a NanoMill™-01 mill in the order listed: 26.1 g of 0.5 mm polystyrene milling media, 2.25 g of lactam compound of formula Ia, 10.1 g of filtered PVP K13-19 polymer stock solution, and 10.1 g of a filtered sodium deoxycholate stock solution. The mill was assembled, and the milling chamber was equilibrated to a temperature of 4-5° C. by means of a circulating thermostatted water bath. The contents were milled for 2 minutes at 1800 rpm, followed by 58 minutes at 2930 rpm. The resulting nanoparticle dispersion containing crystals of lactam compound Ia was separated from the beads by aspirating into a 3-mL sub-Q syringe fitted with a 26 gauge ⅝ needle, and transferred to a suitable vial. Yield=9.2 g (˜41 wt. %). The nanoparticle dispersion was diluted to 2 wt. % drug using a filtered vehicle consisting of 2 wt. % polyvinylpyrrolidone and 0.2 wt. % sodium deoxycholate in Water for Injection.

Lyophilization: One milliliter aliquots of the 2 wt. % nanoparticle dispersion of Example 2 were filled into 10 mL glass vials. The vials were partially stoppered with 20 mm butyl rubber stoppers. The contents of the vial were lyophilized in a Virtis Genesis Model 25EL freeze dryer over a period of 48 hours using the following cycle. The shelf fluid temperature was lowered from 5° C. to −40° C. over two hours. The shelf fluid temperature was held at −40° C. for two hours to freeze the product. To begin primary drying, the chamber pressure was reduced to J50 millitorr (mt) and the shelf fluid temperature was increased to −25° C. over one hour. These primary drying conditions of 150 mt chamber pressure and −25° C. shelf fluid temperature were maintained for 20 hours. To begin secondary drying, shelf temperature was increased to 25° C. over four hours. Secondary drying was conducted at a chamber pressure of 150 mt and a shelf fluid temperature of 25° C. for 18 hours. At the end of the lyophilization cycle, chamber pressure was increased to atmospheric using a nitrogen bleed. The vials were fully stoppered, unloaded from the lyophilizer and sealed.

A nanoparticle dispersion containing the nanoparticles of the lactam compound of formula Ia was prepared by constituting the lyophile and further diluting the lactam compound to a concentration of approximately 1 mg/mL with 5% Dextrose Injection.

### Example 3

### Preparation of a Nanoparticle Dispersion Containing 5 wt. % Lactam Compound Ia and 2 wt. % Pluronic™ F108 Surfactant

A stock solution of 5 wt. % Pluronic™ F108 surfactant was prepared in Water for Injection (WFI) and filtered though a 0.22 micron filter. The following materials were introduced into the jacketed 100-mL milling chamber of a NanoMill™-01 mill in the order listed: 48.86 g of 0.5 mm polystyrene milling media, 2.11 g of the lactam compound of formula Ia, 16.78 g of a filtered solution containing 5 wt. % Pluronic™ F108 surfactant, and 23.05 g of WFI. The mill was assembled, and the milling chamber was equilibrated to a temperature of 4-5° C. by means of a circulating thermostatted water bath. The contents were milled for 2 minutes at 1800 rpm, followed by 88 minutes at 2140 rpm. The resulting nanoparticle dispersion was separated from the beads by aspirating into a 3-mL Sub-Q syringe fitted with a 26 gauge ⅝ inch needle, and transferred to a suitable vial. Yield=31 g of nanoparticle dispersion (˜74 wt. %). Mannitol (400 mg) was dissolved in 8 g of the nanoparticle dispersion with stirring, to give 5 wt. % mannitol.

Lyophilization: Aliquots (˜600 microliter) of the nanoparticle dispersion of Example 3 with and without added mannitol were filled into 10 mL glass vials. The vials were partially stoppered with 20 mm Omniflex Plus RFS stoppers which had been previously sterilized and dried. The product was lyophilized in a Virtis Genesis Model 25EL freeze dryer over a period of 24 hours using the following cycle. The shelf fluid temperature was lowered from 5° C. to −40° C. over two hours. The shelf fluid temperature was held at −40° C. for two hours to freeze the product. To begin primary drying, the chamber pressure was reduced to 150 millitorr (mt) and the shelf fluid temperature was increased to −10° C. over three hours. These primary drying conditions of 150 mt chamber pressure and −10° C. shelf fluid temperature were maintained for 8 hours. To begin secondary drying, the shelf temperature was increased to 25° C. over three hours. Secondary drying was conducted at a chamber pressure of 150 mt and a shelf fluid temperature of 25° C. for 7 hours. At the end of the lyophilization cycle, chamber pressure was increased to atmospheric using a nitrogen bleed. The vials were fully stoppered, unloaded from the lyophilizer and sealed.

### Example 4

### Preparation of a Nanoparticle Dispersion Containing 9.9 wt. % Lactam Compound Ia+2.5 wt. % Human Serum Albumin

Into a 20 ml screw-cap amber glass bottle were introduced, in the following order, 7.5 mL of 0.8 mm YTZ (yttrium stabilized zirconium) ceramic beads (measured with a graduated cylinder), 375 mg of the lactam compound of formula Ia, 310 microliters (˜0.330 g) of 30 wt. % human serum albumin solution, and 3.089 g of deionized water. The capped bottle was placed on a jar mill and rotated at ˜130 rpm for about 17 hours at ambient laboratory temperature (approximately 20° C.). The resulting nanoparticle dispersion was separated from the beads by aspirating into a 3-mL Sub-Q syringe fitted with a 26 gauge ⅝ inch needle, and transferred to a suitable vial. Yield=2.64 g (˜70 wt. %).

### Example 5

### Preparation of a Nanoparticle Dispersion Containing 10.2 wt. % Lactam Compound Ia and 1.9 wt. % PEGylated Phospholipid

A stock solution of 5 wt. % PEGylated phospholipid (1,2-dipalmitoyl-sn-glycero-3-phosphoethanolamine-N-[methoxy(polyethylene glycol)-2000] ammonium salt powder) was prepared in deionized water and filtered through a 0.45 filter. Into a 20 ml screw-cap amber glass bottle were introduced, in the following order, 7.5 mL of 0.8 mm YTZ (yttrium stabilized zirconium) ceramic beads (measured with a graduated cylinder), 375 mg of the lactam compound of Formula Ia, 1.406 g of 5 wt. % PEGylated phospholipid stock solution, and 1.877 g of deionized water. The capped bottle was placed on a jar mill and rotated at ˜130 rpm for about 40 hours at ambient laboratory temperature. Additional milling up to 67 hours was found not to affect the particle size distribution significantly. The resulting nanoparticle dispersion was separated from the beads by aspirating into a 3-mL Sub-Q syringe fitted with a 26 gauge ⅝ inch needle, and transferred to a suitable vial. Yield=2.84 g (˜76 wt. %).

### Example 6

### Preparation of a Nanoparticle Dispersion Containing 10.2 wt. % Lactam Compound Ia, 0.5 wt. % Pluronic™ F68 Surfactant, and 0.025 wt. % Sodium Deoxycholate

Stock solutions of 5 wt. % Pluronic™ F68 surfactant and 1 wt. % sodium deoxycholate were prepared in deionized water and filtered through a 0.45 micron filter. Into a 20 ml screw-cap amber glass bottle were introduced, in the following order, 7.5 mL of 0.8 mm YTZ (yttrium stabilized zirconium) ceramic beads (measured with a graduated cylinder), 375 mg of the lactam compound of formula Ia, 375 microliters of a filtered solution containing 5 wt. % Pluronic™ F68 filtered stock solution, 94 microliters of 1 wt. % sodium deoxycholate filtered stock solution, and 2.906 mL of deionized water. The capped bottle was placed on a jar mill and rotated at ˜130 rpm for about 17 hours at ambient laboratory temperature. The resulting nanoparticle dispersion was separated from the beads by aspirating into a 3-mL Sub-Q syringe fitted with a 26 gauge ⅝ inch needle, and transferred to a suitable vial. Yield=1.53 g (˜41 wt. %).

### Example 7

### Preparation of a Nanoparticle Dispersion Containing 10 wt. % Lactam Compound Ia, 5 wt. % Pluronic™ F68 Surfactant, and 0.25 wt. % Sodium Deoxycholate

Stock solutions of 5 wt. % Pluronic™ F68 surfactant and 1 wt. % sodium deoxycholate were prepared in deionized water and filtered though a 0.2 micron filter.

The following materials were added into the jacketed 10-mL milling chamber of a NanoMill™ mill in the following order: 5.43 g of 0.5 mm polystyrene milling media, 0.466 g of lactam compound of formula Ia, 0.466 mL of a filtered solution of 5 wt. % Pluronic™ F68 surfactant, 1.165 mL of 1 wt. % sodium deoxycholate filtered stock solution, and 2.561 mL of deionized water. The mill was assembled, and the milling chamber was equilibrated to a temperature of 5° C. by means of a circulating thermostatted water bath. The contents were milled for 2 minutes at 1800 rpm, followed by 28 minutes at 5500 rpm. The resulting nanoparticle dispersion was separated from the beads by aspirating into a 3-mL Sub-Q syringe fitted with a 26 gauge ⅝ inch needle, and transferred to a suitable vial. Yield=3.04 g (˜65 wt. %).

Aliquots of the 10 wt. % nanoparticle dispersion were diluted to 2.5 wt. % using deionized water, 10 wt. % mannitol solution, 10 wt. % dextran solution, 10 wt. % trehalose solution, or 10 wt. % sucrose solution without adversely affecting the particle size distribution initially, or after storage for 24 hours under refrigerated conditions (2-8° C.). Additionally, an acceptable cake was obtained by lyophilization.

### Example 8

### Preparation of a Nanoparticle Dispersion Containing 5.4 wt. % Lactam Compound Ia, 0.5 wt. % Pluronic™ F68 Surfactant, 0.27 wt. % Sodium Deoxycholate, and 0.6 wt. % Phospholipon™ 90G Stabilizer

A stock solution of 1 wt. % sodium deoxycholate was prepared in deionized water and filtered through a 0.45 micron filter. Into a 20 ml screw-cap amber glass bottle were added, in the following order, 7.5 mL of 0.8 mm YTZ (yttrium stabilized zirconium) ceramic beads (measured with a graduated cylinder), 187.4 mg of the lactam compound of formula Ia, 18.8 mg of Pluronic™ F68 surfactant (added as a solid), 943 microliters of 1 wt. % sodium deoxycholate filtered stock solution, 22.5 mg of solid Phospholipon™ 90G stabilizer (American Lecithin Co., CT), and 2.271 mL of deionized water. The capped bottle was placed on a jar mill and rotated at ˜130 rpm for about 40 hours at ambient laboratory temperature. The resulting nanoparticle dispersion was separated from the beads by aspirating into a 3-mL Sub-Q syringe fitted with a 26 gauge ⅝ inch needle, and transferred to a suitable vial. Yield=1.53 g (˜41 wt. %).

TABLE 1
Nanoparticle Dispersions of Examples 1 to 8

| | | | D₅₀ | D₉₀ |
|---|---|---|---|---|
| | Lactam | | Average | Average |
| Ex- | Compound | | Particle | Particle |
| am- | Ia | | Diameter | Diameter |
| ple | (wt. %) | Stabilizers | (micron) | (micron) |
| 1 | 5 | 2 wt. % Pluronic ™ F108 | 0.283 | 0.441 |
| | | surfactant | | |
| 2 | 10 | 1.8 wt. % PVP; 0.2 wt. % | 0.188 | 0.252 |
| | | sodium deoxycholate | | |
| 3 | 5 | 2 wt. % Pluronic ™ F108 | 0.102 | 0.256 |
| | | surfactant | | |
| 4 | 9.9 | 2.5 wt. % human serum | 0.136 | 0.229 |
| | | albumin | | |
| 5 | 10.2 | 1.9 wt. % PEGylated | 0.089 | 0.298 |
| | | phospholipid | | |
| 6 | 10.2 | 0.5 Pluronic ™ F68 | 0.289 | 0.466 |
| | | surfactant; 0.025 wt. % | | |
| | | sodium deoxycholate | | |
| 7 | 10 | 0.5 Pluronic ™ F68 | 0.259 | 0.379 |
| | | surfactant; 0.25 wt. % | | |
| | | sodium deoxycholate | | |
| 8 | 5.4 | 0.5 wt. % Pluronic ™ F68 | 0.173 | 0.260 |
| | | surfactant; 0.27 wt % | | |
| | | sodium deoxycholate; 0.6 | | |
| | | wt. % Phospholipon 90G | | |
| | | stabilizer | | |

### Example 9

### Pharmacokinetic Study in Rats

The pharmacokinetic study of nanoparticle dispersions comprising the lactam compound Ia was conducted in rats. The nanoparticle dispersions were prepared as follows:

### Example F1

A nanoparticle dispersion comprising 50 mg/mL lactam compound Ia, 5 mg/mL Pluronic™ F68 surfactant, and 2.5 mg/mL sodium deoxycholate was prepared in water according to the general procedure of Example 7. The nanoparticle dispersion was then diluted to a concentration of 1 mg/ml of the lactam compound Ia with a vehicle comprising 5 mg/mL Pluronic™ F68 surfactant dissolved in 5% Dextrose Injection USP aqueous solution. The final concentrations of the nanoparticle dispersion of Example F1 was 1 mg/mL of the lactam compound Ia, 5 mg/mL Pluronic™ F68 surfactant, 0.06 mg/mL sodium deoxycholate, and 49 mg/mL dextrose in water; and had an average particle diameter (D₅₀) of 403 nm, a D₉₀ value of 1.2 microns, and a D₁₀₀ value of less than 11.6 microns.

### Example F2

A nanoparticle dispersion comprising 50 mg/mL of lactam compound Ia and 20 mg/mL Pluronic™ F108 surfactant was prepared in water according to the general procedure of Example 1. Approximately 0.2 mL of the nanoparticle dispersion was transferred to a vial and lyophilized using the general lyophilization procedure of Example 3 (without mannitol). The lyophile was constituted with 10 mL of Normal Saline (0.9% NaCl) to obtain the nanoparticle dispersion of Example F2, which comprised 1 mg/mL of the lactam compound Ia, 0.4 mg/mL Pluronic™ F108 surfactant, and 9 mg/mL sodium chloride in water; and had an average particle diameter (D₅₀) of 290 nm, a D₉₀ value of 1.3 microns, and a D₁₀₀ value of less than 34 microns.

### Example F3

A nanoparticle dispersion comprising 100 mg/mL of the lactam compound Ia and 25 mg/mL of human serum albumin was prepared in water according to the general procedure of Example 4. The nanoparticle dispersion was then diluted to a concentration of 1 mg/mL of the lactam compound Ia with 5% Dextrose Injection USP aqueous solution. The nanoparticle dispersion of Example F3 comprised 1 mg/mL of the lactam compound Ia, 25 mg/mL human serum albumin, and 49.5 mg/mL dextrose in water; and had an average particle diameter (D₅₀) of 147 nm, a D₉₀ value of 228 nm, and a D₁₀₀ value of less than 766 nm.

### Example F4

A nanoparticle dispersion comprising 100 mg/mL of the lactam compound Ia, 20 mg/mL PEGylated phospholipid was prepared in water according to the general procedure of Example 5, except that the milling process employed a NanoMill™-01 mill with a milling time of approximately 82 minutes instead of the low energy milling described in Example 5. The nanoparticle dispersion was then diluted to a concentration of 1 mg/mL of the lactam compound Ia with 5% Dextrose Injection USP aqueous solution. The nanoparticle dispersion of Example F4 comprised 1 mg/mL of the lactam compound Ia, 0.2 mg/ml PEGylated phospholipid, and 49.5 mg/mL dextrose in water; and had an average particle diameter (D₅₀) of 241 nm, a D₉₀ value of 401 nm, and a D₁₀₀ value of less than 3.4 microns.

Control Solution: A solution of the lactam compound Ia was prepared by dissolving 16 mg of lyophilized lactam compound Ia into 4 mL of a Cremophor EL/ethanol solution (50/50), and diluting 3 mL of the resulting solution with 9 mL of Normal Saline (0.9% NaCl). The control solution comprised 1 mg/mL lactam compound Ia, 12.5% Cremophor EL, 12.5% ethanol, and 6.75 mg/mL sodium chloride in water (75%).

### Pharmacokinetics in Rats

The nanoparticle dispersions of Examples F1 to F4 and the control solution were administered intravenously to male sprague-dawley rats to evaluate the pharmacokinetics of the lactam compound Ia. The lactam compound Ia was administered at a dose of 2 mg/kg for each formulation.

The study design was a single-dose, four-treatment, two-period non-randomized, crossover design. The lactam compound Ia was administered as a single IV bolus dose to male rats. Animals were divided into two groups during each period. Each group consisted of rats that received one of the nanoparticle dispersions (n=7) or the control solution (n=2). The study design is shown in Table 2.

TABLE 2
Design of Pharmacokinetic Study in Rats

| Period | Treatment | Dose (mg/kg) | Number of Rats |
|---|---|---|---|
| 1 | F3 | 2 | 7 |
| 1 | F4 | 2 | 7 |
| 2 | F1 | 2 | 7 |
| 2 | F2 | 2 | 7 |
| 1 and 2 | Control solution | 2 | 8^{a} |

a Two rats were treated with the control solution along with each nanoparticle dispersion.

### Sample Collection and Analysis

Blood samples were collected from the jugular or saphenous vein of each animal at 0.033, 0.025, 0.5, 0.75, 1, 2, 4, 6, 8, 12, and 24 hours after dosing. Samples were collected into tubes containing EDTA as an anticoagulant. Plasma was obtained following centrifugation. Concentrations of the lactam compound Ia in plasma were determined using a validated LC/MS/MS method (range=2 to 500 ng/mL).

### Pharmacokinetic Analysis

The pharmacokinetic parameters AUC_{0-24 hrs}, Cₘₐₓ, CL, V_{d}, and t_{1/2} were calculated using noncompartmental methods by eToolbox/Kinetica (Version 2.4, InnaPhase Corporation, Philadelphia, Pa.). Values below LLOQ were not used in calculations. AUC was calculated using the trapezoidal rule.

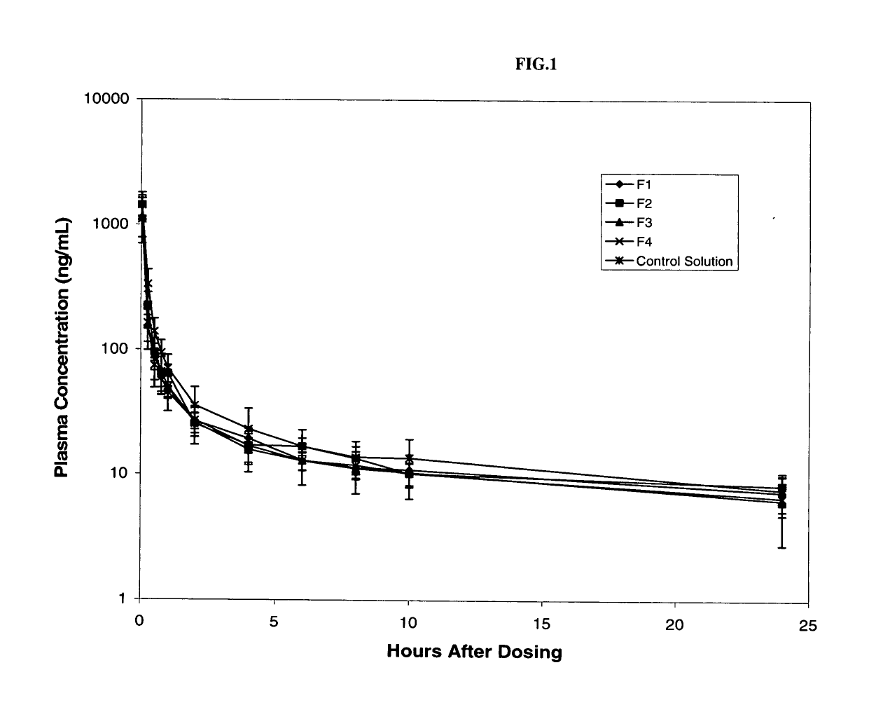

The plasma concentrations and the pharmacokinetic parameters of the lactam compound Ia in the rats following a single IV bolus administration of the nanoparticle dispersions F1 to F4 and the control solution are shown in FIG. 1 and Table 3, respectively. The mean terminal half-life ranged from approximately 20 to 29 hours. the volume of distribution was greater than the volume of the central compartment, indication distribution of the lactam compound Ia into tissues.

TABLE 3
Pharmacokinetic Parameters of Examples F1 to F4 and Control Solution

| | Cₘₐₓ | AUC_{0-24 hrs} | t_{1/2} | CL | V_{d} |
|---|---|---|---|---|---|
| Parameter | (ng/mL) | (ng · h/mL) | (h) | (mL/min) | (L) |
| F1 | 1413 ± 238 | 559 ± 56 | 29.1 ± 13.1 | 22.2 ± 4.9 | 52.2 ± 16.0 |
| F2 | 1330 ± 213^{a} | 550 ± 46^{a} | 21.6 ± 6.8^{b} | 24.7 ± 3.4^{b} | 45.8 ± 14.5^{b} |
| F3 | 1169 ± 457 | 498 ± 175 | 20.2 ± 10.0 | 33.5 ± 16.1 | 48.1 ± 8.2 |
| F4 | 1103 ± 249 | 481 ± 63 | 19.5 ± 9.2 | 28.9 ± 5.9 | 45.9 ± 14.1 |
| Control | 1436 ± 279 | 682 ± 150 | 23.5 ± 13.8 | 20.5 ± 4.9 | 38.9 ± 18.1 |
| Solution^{c} | | | | | |

n = 7 for calculation of mean and SD, except where indicated.
^{a}n = 6.
^{b}n = 5.
^{c}n = 8 (data from 2 experiments were pooled).

The statistical analysis indicated that no significant differences exists in the pharmacokinetic parameters AUC_{0-24 hrs}, CL, and V_{d} among the different formulations, except that AUC_{0-24 hrs} was statistically different (1.41-fold, p<0.01) between Example F4 and the control solution.

1. A nanoparticle dispersion comprising:
i) nanoparticles having an average particle diameter of less than about 1 micron and which comprise at least one lactam compound having the formula: 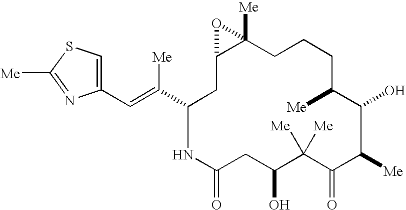
ii) at least one stabilizer; and
iii) a liquid medium;
wherein;
said nanoparticles are dispersed in said liquid medium; and
said at least one stabilizer is adsorbed on surfaces of said nanoparticles in an amount sufficient to provide said nanoparticles with the average particle diameter of less than about 1 micron.

2. (canceled)

3. The nanoparticle dispersion according to claim 1 comprising up to about 40 weight % of said nanoparticles, based on weight of said nanoparticle dispersion.

4. The nanoparticle dispersion according to claim 1 comprising from about 0.1 to about 10 weight % of said at least one stabilizer, based on weight of said nanoparticle dispersion.

5. The nanoparticle dispersion according to claim 1 wherein said liquid medium is an aqueous medium.

6. The nanoparticle dispersion according to claim 5 wherein said at least one stabilizer is selected from the group consisting of sodium deoxycholate, polyvinylpyrrolidone, albumin, polyethylene glycol-phospholipids, lecithin, and block copolymers of ethylene oxide and propylene oxide.

7. The nanoparticle dispersion according to claim 5 wherein said aqueous medium has a pH in a range of from about 4 to about 9.

8. The nanoparticle dispersion according to claim 5 comprising, based on weight of said nanoparticle dispersion:
i) from about 0.1 to about 40 weight % of said nanoparticles comprising said lactam compound of formula I having formula: 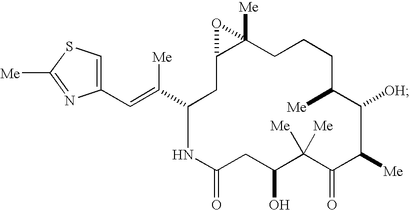
and
ii) from about 0.1 to about 10 weight % of said at least one stabilizer;
wherein said aqueous medium has a pH in a range of from about 6 to about 8.

9. A process for making a solid nanoparticulate composition comprising: nanoparticles comprising at least one lactam compound 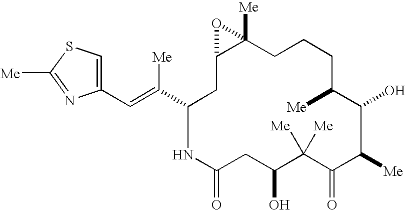
wherein:
said nanoparticles have an average particle diameter of less than about 1 micron; wherein said process comprises providing the nanoparticle dispersion according to claim 1 and removing the liquid medium therefrom.

10. A solid nanoparticulate composition prepared according to claim 9.

11. (canceled)

12. A method for treating cancer or other proliferative diseases in a mammal, comprising: administering an effective amount of a pharmaceutical composition to said mammal, wherein said pharmaceutical composition comprises a nanoparticle dispersion according to claim 1.

13. The method according to claim 12 wherein said pharmaceutical composition is administered intravenously.

14. The method according to claim 12 wherein said cancer is breast cancer, prostate cancer, or lung cancer.

15. The method according to claim 12 wherein said lactam compound of formula I is:

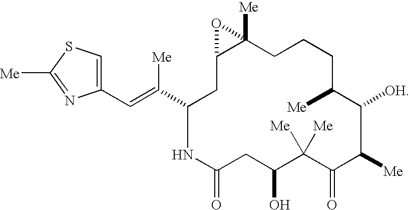

16-18. (canceled)

19. A solid nanoparticulate composition comprising the lactam compound having the formula, 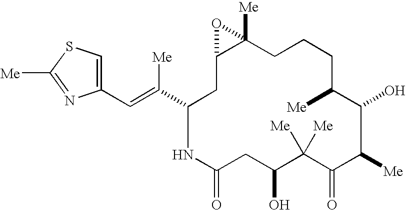
wherein said solid nanoparticulate composition is prepared by providing the nanoparticle dispersion according to claim 1 and removing the liquid medium therefrom.
